(19) United States
(12) Patent Application Publication
(10) Pub. No.: US 2010/0055056 A1
(43) Pub. Date: Mar. 4, 2010

(54) AGENT FOR IMPROVING PERMEATION OF A DRUG INTO A NAIL AND AN EXTERNAL TREATING AGENT CONTAINING THE SAME

(75) Inventors: Masatoshi KATO, Shinagawa-ku (JP); Hiroyuki KUSAKABE, Shinagawa-ku (JP); Kazuhiro NIMURA, Shinagawa-ku (JP); Hiroaki KIMURA, Shinagawa-ku (JP); Koh NAGASAWA, Shinagawa-ku (JP); Akihiro HASHIGUCHI, Shinagawa-ku (JP); Chisato HIROSAWA, Shinagawa-ku (JP); Tomohiro TADA, Shinagawa-ku (JP)

Correspondence Address: SUGHRUE MION, PLLC, 2100 PENNSYLVANIA AVENUE, N.W., SUITE 800, WASHINGTON, DC, 20037, US

(73) Assignee: SATO PHARMACEUTICAL CO., LTD., Tokyo (JP)

(21) Appl. No.: 12/204,469

(22) Filed: Sep. 4, 2008

(51) Int. Cl. A61K 31/137; C07D 233/54; C07D 249/04; C07D 249/08; C07D 213/02

(52) U.S. Cl. 424/61; 548/335.1; 548/255; 548/262.2; 546/1

(57) ABSTRACT

The present invention herein provides a compound which promotes drug-permeation into nails. A penetrating promoter comprises, for instance, the following formulas.

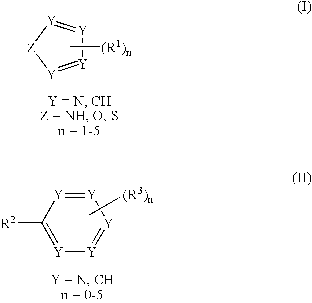

R¹, R² and R³ are alkyl groups having a carbon number of 1 to 15 and have OH groups at their ends.

### TECHNICAL FIELD

The present invention relates to an agent for improving or promoting the permeation or infiltration of a drug into a nail and a nail-treating agent, for external use, containing the permeation improver.

### BACKGROUND ART

Various kinds of agents have widely been used for the administration or application of drugs through the transdermal route, but the achievement of any sufficient effect of such transdermal administration cannot always be expected in case of the drugs having a low absorbability. The skin comprises epidermis, corium and subcutaneous tissue (subcutis) and the epidermis can further be divided into the following four layers, in the order from the top of the skin, the stratum corneum epidermidis (horny layer), the stratum granulosum epidermidis (granular layer), the stratum spinosum epidermidis (prickle cell layer) and the stratum basale epidermidis (basal layer). Among them, the horny layer comprises keratin and fibrous proteins, as well as additional components such as ceramide and neutral lipids, and it serves to prevent the permeation of any external foreign matters and the escape of the moisture in the skin and/or the body through evaporation.

The barrier action peculiar to the skin would constitute a cause for the prevention of the transdermal absorption of a drug. As a means for eliminating this problem, one can employ, for instance, a method which makes use of a permeation-improving agent.

There have already been proposed a variety of permeation-improving agents and specific examples thereof include dimethyl sulfoxide, N,N-dimethylformamide, urea, and fatty acids and esters thereof represented by lauric acid and diethyl sebacate, azacycloalkane-2-one derivatives including Azone (Patent Document 1 specified below), dioxolane derivatives (Patent Document 2 specified below), and macrocyclic esters (Patent Document 3 specified below).

However, these permeation-promoters are still insufficient since they suffer from problems such as their low stability and skin-irritant properties thereof and the permeation-improving effect thereof is not sufficient at all. Accordingly, there has not yet been developed any effective drug-permeation promoter which can be put into practical use.

The foregoing problems would be more conspicuous in case of the nail. The nail is one of appendages of the skin, it is rich in keratin like the skin, but the keratin component present in the horny layer of the skin is soft keratin, while that of the nail is hard keratin. For this reason, the nail has a high content of cystine and has a complicated structure in which polypeptide chains are crosslinked through disulfide bonds. In addition, the thickness of the nail is about 100 times higher than that of the horny layer of the skin and therefore, the nail in general has a significantly low drug-permeability as compared with that observed for the skin. As a typical disease of such nails, the ringworm of the nail or tinea unguium can be listed.

The ringworm of the nail is a superficial mycosis caused through the parasitism of Trychophytom on the nail like the tinea pedis and the parasitism thereof causes an increase in the thickness of the nail, it makes the nail opaque and the ringworm of the nail is sometimes accompanied by pains. Methods for treating such ringworm of the nail presently known and mainly adopted in the field of the dermatology are, for instance, those comprising orally administering such oral drugs as griseofulvin or itraconazole; and those which comprise the step of declawing through surgical operations.

The former method would force, on patients, the use of such a drug over a long period of time and therefore, suffers from the problems of side effects such as hepatopathy and the interaction with other drugs, while the latter method suffers from a problem in that the declawing would be accompanied by severe pains and that a part of the fragile nail remains unremoved at the affected site. For this reason, the ringworm of the nail should desirably be treated by the local administration of a drug for external use. As has been discussed above, however, the nails per se serve as a strong barrier and prevent the absorption of any drug and accordingly, any desired effect cannot be expected even when such a drug is applied onto the affected portions over a long period of time.

Thus, the treatment of the ringworm of the nail is quite difficult as has been described above and there has not yet been established any safe and effective method for treating the ringworm of the nail.

Under such circumstances, many attempts have been done for the improvement of the effect of the topical treatment. For instance, there may be listed a method which makes use of a combination of an antifungal agent with a fatty acid (Patent Document 4 specified below), urea (Patent Document 5 specified below) or a basic substance (Patent Document 6 specified below), but the use of such combinations of drugs is quite uncomfortable and they are insufficiently adhered to the nails. Accordingly, any satisfactory effect cannot be anticipated. Further, there has been proposed a method which makes use of a film-forming agent for the improvement of the adhesion of such a drug to the nail (Patent Documents 7 to 9 as will be specified below), but none of them ensures the achievement of any satisfied effect at all since they are still insufficient in the ability of releasing the drug from the resulting film and in the penetrability of the drug into the nail, although they can improve the adhesion of the drug to the nail to some extent. In the first place, the nail has a three-layer structure comprising, from the top, an upper layer, a middle layer and a lower layer of the nail plate and the tissues behind the nail plate called nail matrix (matrix unguis) are brought into contact with the nail plate. It has been known that the lower layer of the nail plate and the nail matrix are infected with Trychophytom as the causative fungi of the ringworm of the nail at a high probability and accordingly, it would be quite important to deliver a desired antifungal agent to the lower layer of the nail plate and the nail matrix in order to make, effective, the pharmacotherapy using a topical drug for external use. However, the nail serves as a strong barrier and therefore, the drug cannot reach the desired affected sites having a high rate of infection with the fungi and this in turn becomes a cause of poor effect of the treatment.

In addition, there has also been proposed the use of an antifungal agent and an N-alkyl heterocyclic compound in combination (Patent Document 10 as will be specified below). However, the N-alkyl heterocyclic compound disclosed in Patent Document 10 is not a compound carrying a hydroxyl group at the terminal thereof, but is a compound in which the alkyl group is directly bonded to an N atom of the heterocyclic ring. Moreover, in the N-alkyl heterocyclic compound disclosed in Patent Document 10, the carbon atom number of the alkyl group is limited to a specific range and Patent Document 10 neither discloses nor suggests the compounds carrying alkyl groups other than those specified therein, although the compounds disclosed in Patent Document 10 are those in which the alkyl group is directly bonded to an N atom of the heterocyclic ring. Further, the compounds disclosed in this Patent Document are never inspected for their effects as permeation-promoters.
Patent Document 1: JP-A-52-1035;
Patent Document 2: JP-A-1-135727;
Patent Document 3: U.S. Pat. No. 5,023,252;
Patent Document 4: JP-A-58-32818;
Patent Document 5: JP-A-63-258814;
Patent Document 6: JP-A-2004-83439;
Patent Document 7: JP-A-62-155205;
Patent Document 8: JP-A-2-264708;
Patent Document 9: WO 96/11710;
Patent Document 10: JP-A-2000-5 16928.

### DISCLOSURE OF THE INVENTION

### Subject to be Attained by the Invention:

As has been discussed above in detail, there have variously been investigated drug-permeation improvers in order to deliver a desired drug even to the intended sites of the nail having a quite low drug-permeability, but the permeation-improving effects of these promoters are still unsatisfied and the permeation-improving agent in itself suffers from a number of problems such that it is unstable and uncomfortable when using the same and has irritant properties. For this reason, there has been desired for the development of a widely applicable drug-permeation promoter. Accordingly, it is an object of the present invention to provide a drug-permeation promoter which is highly safe, has excellent stability and shows a more excellent effect when applied to a drug for treating nails as well as a nail-treating agent, for external use, containing the promoter.

### Means for Attaining the Subject:

Thus, the inventors of this invention have conducted various studies to develop a drug-permeation promoter excellent in the effect of improving the drug-permeation into the nail while taking into consideration the drawbacks associated with the conventionally proposed drug-permeation promoters, have found that a nitrogen atom-containing heterocyclic compound shows an excellent effect of improving drug-permeation into the nail and have thus completed the present invention.

Thus, the present invention herein provides a drug-permeation improving agent comprising a compound represented by the following Formula (I) or a pharmaceutically acceptable salt thereof:

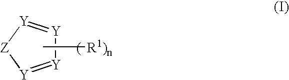

(In the formula (I),

Z represents a nitrogen atom (—NH—), an oxygen atom (—O—) or a sulfur atom (—S—);

Y represents a tertiary nitrogen atom (—N═) or a tertiary carbon atom (—C═), R¹ represents a C1 to C15 saturated or unsaturated hydrocarbon group [except for the compounds of Formula (I) in which R¹ is a saturated hydrocarbon group represented by CH₃CₘH₂ₘ wherein m is a number ranging from 5 to 14 and it is directly bonded to the nitrogen atom on the 5-membered ring in Formula (I)]; and

n is a number ranging from 1 to 5);

as well as a nail-treating agent for external use, which comprises the compound of Formula (I) and/or the salt thereof; or a permeation-improving agent; or

a drug-permeation promoter comprising a compound represented by the following Formula (II) or a pharmaceutically acceptable salt thereof:

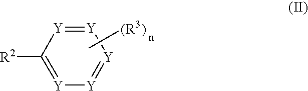

### (In Formula (II),

R² and R³ independently represent a C1 to C15 saturated or unsaturated hydrocarbon group or a C1 to C15 saturated or unsaturated hydrocarbon group connected to the heterocyclic ring through an oxygen atom;

Y represents a tertiary nitrogen atom (—N═) or a tertiary carbon atom (—C═); and

n is a number ranging from 0 to 5);

as well as a nail-treating agent for external use, which comprises the foregoing compound of Formula (II) and/or salt thereof.

### BEST MODE FOR CARRYING OUT THE INVENTION

In the compounds of Formulas (I) and (II) used as principal components according to the present invention, the hydrocarbon group represented by R¹, R² or R³ is a saturated hydrocarbon group or an unsaturated hydrocarbon group carrying one or more unsaturated bonds selected from double and triple bonds. The hydrocarbon group is suitably an alkyl or alkenyl group having 1 to 15, preferably 2 to 10 and more preferably 3 to 8 carbon atoms, with an alkyl group being particularly preferred. The alkyl group may be linear, branched or circular one.

The hydrocarbon group represented by R² is suitably one having 1 to 15, preferably 2 to 10 and more preferably 3 to 8 carbon atoms.

The hydrocarbon group represented by R³ is suitably one having 1 to 15, preferably 2 to 10 and more preferably 3 to 8 carbon atoms, with an alkyl group being particularly preferred. Alternatively, the substituent R³ may likewise be an alkoxy group. In the alkoxy group, the alkyl moiety may be the same as that defined above in connection with the substituent R². Moreover, when the substituent R³ represents an alkoxy group, the latter may have a hydroxyl group at the terminal thereof and further one which forms an ether bond with a divalent alkylene glycol such as ethylene glycol or propylene glycol through the hydroxyl group. In case where an alkylene glycol is added to or bonded with the alkyl group, the added molar number of the alkylene glycol is suitably on the order of 1 to 5 and preferably 1 to 3.

The alkyl group may have one or a plurality of substituents and when it has a plurality of substituents, these substituents may be the same or different.

Examples of such substituents suitably used in the invention are halogen atoms (such as F, Cl, Br, I), hydroxyl or hydroxide group (—OH), amino groups, alkoxy groups, carbonyl groups, formyl groups, carboxyl groups, carboxyester groups, carboxyamido groups, ureido groups, phenyl groups, and aromatic heterocyclic groups. Particularly preferred are those having, on the terminal carbon atom, a hydroxyl group, an alkoxy group, a carboxyl group, a carboxyester group or the like, in particular, a hydroxyl group. Moreover, when the foregoing compound has a nitrogen atom-containing 5-membered ring, the hydrocarbon group may or may not be directly bonded to the nitrogen atom. In this case, however, the hydrocarbon group having a hydroxyl group as a substituent is particularly preferably directly bonded to the nitrogen atom.

The foregoing alkoxy group is a group consisting of an alkyl group and an oxygen atom. In this connection, the alkyl group may be the same as those discussed above. Preferably, the alkyl moiety of the alkoxy group is one suitably having 1 to 8, in particular, 1 to 5 carbon atoms.

The foregoing amino group is one represented by the formula: —NR⁴R⁵, wherein R⁴ and R⁵ represent a hydrogen atom or an alkyl group identical to that specified above.

The foregoing carboxyester group is one represented by the formula: —COOR⁶, in which R⁶ represents an alkyl group identical to that specified above.

The foregoing carboxyamido group is one represented by the formula: —CONR⁷R⁸, in which the substituents R⁷ and R⁸ present in the group specified by the formula NR⁷R⁸ represent a hydrogen atom or an alkyl group identical to that specified above.

The aforementioned ureido group is one represented by the formula: —NR⁹CONR¹⁰R¹¹, wherein the substituent R⁹ and the group NR¹⁰R¹¹ are the same as those defined above in connection with the alkyl group and the amino group, respectively.

The aforementioned phenyl group may have a substituent and the substituent may be the same as that defined above.

The aforementioned aromatic heterocyclic group suitably used herein are, for instance, imidazolyl, triazolyl, pyridyl, pyrrolidyl, furyl and thiophenyl groups and these heterocyclic groups may have a substituent. Examples of such substituents are the same as those specified above.

When the foregoing 5-membered and 6-membered rings have hydrocarbon groups as the substituents R¹, R² and R³, and when the number of such hydrocarbon groups is not more than 4 for the 5-membered ring or not more than 5 for the 6-membered ring, these rings may be substituted with substituents other than a hydrocarbon group, at the remaining positions to be substituted. Examples of such substituents suitably used herein are halogen atoms, amino groups, cyano groups, nitro groups, hydroxyl groups, and alkoxy groups. The halogen atom, amino group, and alkoxy group are the same as those described above, respectively.

The compounds used in the drug-permeation promoter according to the present invention are preferably those represented by the following Formulas (I-1) to (I-4):

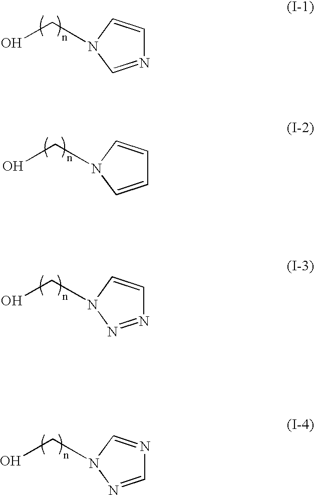

In the foregoing Formulas (I-1) to (I-4), n is preferably an integer ranging from 3 to 10, and preferably 5 to 7. The hydroxyl group thereof may be bonded to an alkyl group identical to that specified above to thus form an alkoxy group.

The compound carrying a 6-membered ring represented by Formula (II) is suitably one in which the nitrogen atom as a hetero atom is arranged at the 2-, 3- or 4-position with respect to the position on the ring substituted with R², in particular, one having the hetero atom at the 4-position thereof. The hydrocarbon group represented by R² may be linked to the 6-membered ring directly or through an oxygen atom. In this connection, the number specifying the position of each specific substituent of the foregoing compound is herein simply given for convenience and it is not controlled by the IUPAC nomenclature at all.

Specific examples of such 6-membered compounds suitably used in the present invention include those represented by the following Formulas (II-1) to (II-3):

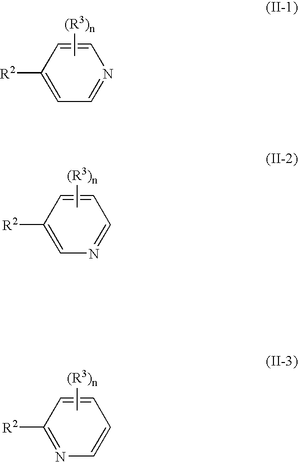

In these Formulas (II-1) to (II-3), R² may suitably be a group defined above having 3 to 10, preferably 5 to 7 carbon atoms. Particularly preferred are alkyl or alkoxy group, and particularly preferred are ones each carrying a hydroxyl group at the terminal thereof.

The compound represented by Formula (I) or (II) is used, in the nail-treating agent for external use, in an amount, for instance, ranging from 0.5 to 80% by mass and preferably 3 to 20% by mass on the basis of the total mass of the nail-treating agent.

It would be easy for those of ordinary skill in the art to prepare the compounds represented by Formula (I) starting from known raw materials. For instance, they can be prepared according to the known technique as will be detailed below.
Process A (This process can be applied to the preparation of Compounds 1 to 19 listed in the following Table 1):

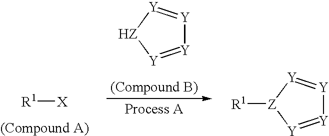

(In the foregoing reaction scheme, R¹, Y and Z are the same as those defined above in connection with Formula (I) and X represents a halogen atom).
Process B (This process can be applied to the preparation of Compounds 20 to 24 listed in the following Table 1):

Alternatively, the compounds of Formula (I) can likewise be prepared according to the same procedures disclosed in the article of Sycheva, T. P. et al.: Chem. Heterocycl. Compd. (Engl. Transl.), CODEN: CHCCAL, 8, <1972> 5-7).
Process C (This process can be applied to the preparation of Compound 25 listed in the following Table 1):

In addition, the compounds of Formula (I) according to the present invention can also be prepared according to the known method which makes use of, for instance, the Paal-Knorr synthetic procedures using γ-diketone.

More specifically, the compound of Formula (I) is prepared according to the process A, or by reacting a compound A and Compound B in an inert solvent in the presence of a base. Examples of Compound A include 8-bromo-1-octanol, 7-bromo-1-heptanol, 6-bromo-1-hexanol, 5-bromo-1-pentanol and bromo-cycloheptane; examples of Compound B are imidazole, 2-methylimidazole, 2-undecylimidazole, pyrrole, pyrazole, 1H-1,2,3-triazole and 1H-1,2,4-triazole; examples of the bases are sodium hydroxide, and potassium carbonate; and examples of the reaction mediums usable herein are dehydrated dimethylformamide, and dehydrated tetrahydrofuran. The reaction can be carried out, for example, by heating the reaction system under the following conditions: a temperature ranging from 60 to 150° C.; a reaction time ranging from 3 to 48 hours to thus give each corresponding Compound (I). The resulting product can be purified by the silica gel column chromatography technique. In addition, the compound (I) thus prepared is, if necessary, further purified through distillation.

It would be easy for those of ordinary skill in the art to prepare the compounds of Formula (II) starting from known raw materials. For instance, the compounds of Formula (II) (such as Compound Nos. 26 to 28 listed in the following Table 4) can be prepared by carrying out the Wittig Reaction and then treating the reaction product by any known method such as hydrogenation.

Furthermore, the compounds of Formula (II) (such as Compound Nos. 29 to 37 listed in the following Table 4) can be prepared by any known method such as the ether-synthesizing method of Williamson.

The effective components suitably used in the present invention include, for instance, an antifungal agent, an antibiotic, an anti-inflammatory agent, a local anesthetic, an anti-allergic agent, an anti-histamic agent, and vitamins. Specific examples of these effective components are as follows: preferably used herein as the antifungal agents include, for instance, nystatin, naftifine, terbinafine, butenafine, amorolfine, clotrimazole, miconazole, econazole, tioconazole, ketoconazole, lanoconazole, neticonazole, fluconazole, itraconazole, ciclopirox, ciclopirox olamine, rilopirox, tolnaftate, griseofulvin, flucytosine, amphotericin B, and 5-FU; preferably used herein as the antibiotics include, for instance, penicillin, meticilin, ampicillin, cephalosporine, cefalexin, streptomycin, gentamicin, kanamycin, tetracycline, minocycline, erythromycin, lincomycin, clindamycin, mikamycin, vancomycin, and chloramphenicol; examples of the anti-inflammatory agents suitably used herein include, but are not limited to steroidal anti-inflammatory agents such as dexamethasone acetate, betamethasone valerate, hydrocortisone butyrate, prednisolone, and fluocinolone acetonide, and non-steroidal anti-inflammatory agents such as aspirin, salicylic acid, acetaminophen, ibuprofen, indometacin, diclofenac, sulindac, colchicines, mefenamic acid, felbinac, and fenbufen; preferably used herein as the local anesthetics include, for instance, lidocaine, dibucaine, and procaine; preferably used herein as the anti-allergic agents are, for instance, ibudilast, tranilast, and sodium salt of cromoglicic acid; preferably used herein as the anti-histamic agents are, for instance, diphenhydramine, diphenylpyrallin, fumaric acid clemastine, chlorpheniramine maleate, and mequitazine; preferably used herein as the vitamins are, for instance, vitamin A, vitamin B1, vitamin B2, vitamin B6, vitamin C, vitamin D, vitamin E, folic acid, lipoic acid, ubiquinone, and inositol. In addition to the foregoing, suitably used herein as the effective components further include, for instance, morphine, nicotine, chondroitin sulfate, and nitroglycerin.

Each of these drug components is used in an appropriate amount selected depending on each specific purpose. For instance, when using, for instance, terbinafine, butenafine, and amorolfine as an antibiotic agent, they are incorporated into the intended nail-treating agent for external use in an amount of, for instance, ranging from 0.1 to 30% by mass and preferably 0.5 to 15% by mass on the basis of the total mass of the nail-treating agent.

The drug-permeation promoter according to the present invention is used as an agent externally applied to the nail and it may be in the form of, for instance, a nail-lacquer, a manicure, a solution, an ointment, a plaster, a gel, a cream, a patch, a poultice, and an aerosol. These preparations can be prepared according to the method known and common to those of ordinary skill in the art, but they may, for instance, be prepared according to the methods disclosed in, for instance, “An Interpretation of The 15^{th} Revised Japanese Pharmacopoeia” (published on June, 2006, by HIROKAWA Publishing Co., Ltd.) and a book on “Methods for Preparing an Agent Externally Applied to the Skin” (written by TAKANO Masahiko and MIYAZAKI Junichi, published on Showa 37, September 20, by NANZANDO Publishing Co., Ltd.). Moreover, in the production of these formulations, there may be used, for instance, bases, solvents, solubilizing agents, film-forming agents, perfumes, coloring agents and containers commonly known to and used by those of ordinary skill in the art, but specific examples thereof are disclosed in, for instance, Dictionary of Medical Additives 2005 (edited by Association of Medical Additives in Japan, Yakuji Nippo Co., Ltd., published on Jul. 12, 2005).

The typical bases usable in the present invention are not restricted to any specific one for the nail lacquer preparation and the manicure preparation and may be various kinds of bases commonly used therein. Examples of such bases are methacrylic acid esters•methacrylic acid copolymers (such as EUDRAGIT L100 available from DEGUSSA Company), copolymers of methacrylic acid esters, cellulose derivatives, acrylic acid•styrene copolymers, ethylene•vinyl acetate copolymers, alkyd resins, polyester resins, polyvinyl alcohols, and soluble nylon, which may be incorporated into the drug-permeation promoter according to the present invention alone or in any combination of at least two of them. In addition, suitably used in the invention as the organic solvents are preferably lower alcohols such as ethanol and isopropanol.

Moreover, the drug-permeation promoter according to the present invention may further comprise, in addition to the foregoing, an auxiliary agent for solubilization, a volatilization-retardant agent, and a plasticizer and suitably used herein include, for instance, ethyl acetate, propyl acetate, butyl acetate, acetone, toluene, triacetin, and menthol.

The base for the ointment is not restricted to any particular one and may be one commonly used in this field. Specific examples thereof are higher fatty acids such as myristic acid, palmitic acid, stearic acid, oleic acid and adipic acid, and further esters thereof; waxes such as bees wax and spermaceti, higher alcohols such as cetanol and stearyl alcohol, various kinds of silicone oils, hydrocarbons such as hydrophilic vaseline, white vaseline, purified lanoline, and liquid paraffins. Moreover, examples of additives suitably used in the invention are a variety of surfactants, humectants, and contact dermatitis-preventing agents.

The bases used in the creams are not limited to any specific one insofar as they have commonly been used for the preparation of them and specific examples thereof suitably used herein are higher fatty acid esters such as myristic acid esters and palmitic acid esters; lower alcohols such as ethanol and isopropanol; polyhydric alcohols such as propylene glycol; higher alcohols such as cetanol; carbohydrates such as squalane; and liquid paraffins.

In addition, additives such as various kinds of emulsifying agents, antiseptic agents and skin fit preventing agents may likewise be incorporated into the cream preparations.

### EXAMPLES

The present invention will hereunder be described in more detail with reference to the following Examples, but the scope of the present invention is not limited to these specific Examples at all.

The compounds (I) represented by Formula (I) specifically synthesized herein are those having each corresponding structure summarized in the following Table 1. In this respect, however, the number used for specifying the position of each specific atom constituting the 5-membered ring (1-position to 5-position) shown in Table 1 is herein simply given for convenience and it is not controlled by the IUPAC nomenclature at all.

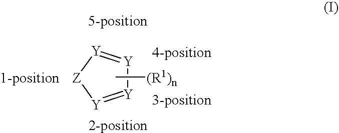

(In this formula, R¹, Y and Z are the same as those defined above in connection with the foregoing general formula (I)).

TABLE 1

| Comp. | | | | | | | |
|---|---|---|---|---|---|---|---|
| No. | Z (1-p) | Y (2-p) | Y (3-p) | Y (4-p) | Y (5-p) | R¹ (1-p) | R¹ (2~5-p) |
| No. 1 | N | CH | N | CH | CH | —(CH₂)₇OH | — |
| No. 2 | N | CH | N | CH | CH | —(CH₂)₃OH | — |
| No. 3 | N | CH | N | CH | CH | —(CH₂)₅OH | — |
| No. 4 | N | CH | N | CH | CH | —(CH₂)₆OH | — |
| No. 5 | N | CH | N | CH | CH | —(CH₂)₈OH | — |
| No. 6 | N | CH | N | CH | CH | —(CH₂)₁₀OH | — |
| No. 7 | N | CH | N | CH | CH | —(CH₂)₅OMe | — |
| No. 8 | N | CH | N | CH | CH | —(CH₂)₆OMe | — |
| No. 9 | N | CH | N | CH | CH | —(CH₂)₇OMe | — |
| No. 10 | N | CH | N | CH | CH | —(CH₂)₈OMe | — |
| No. 11 | N | CH | CH | CH | CH | —(CH₂)₇OH | — |
| No. 12 | N | N | N | CH | CH | —(CH₂)₇OH | — |
| No. 13 | N | N | N | CH | CH | —(CH₂)₁₀OH | — |
| No. 14 | N | N | CH | N | CH | —(CH₂)₇OH | — |
| No. 15 | N | N | CH | N | CH | —(CH₂)₁₀OH | — |
| No. 16 | N | N | CH | CH | CH | —(CH₂)₇OH | — |
| No. 17 | N | CH | N | CH | CH | cycloheptyl | — |
| No. 18 | N | C | N | CH | CH | —(CH₂)₇OH | —Me (2-p) |
| No. 19 | N | C | N | CH | CH | —(CH₂)₇OH | —(CH₂)₁₀Me (2-p) |
| No. 20 | O | C | N | C | CH | — | —(CH₂)₂Me (2-p) |
| | | | | | | | —COOEt (4-p) |
| No. 21 | O | C | N | C | CH | — | —(CH₂)₈Me (2-p) |
| | | | | | | | —COOEt (4-p) |
| No. 22 | O | C | N | C | CH | — | —(CH₂)₈Me (2-p) |
| | | | | | | | —COOH (4-p) |
| No. 23 | O | C | N | CH | CH | — | —(CH₂)₈Me (2-p) |
| No. 24 | O | C | N | CH | C | — | —(CH₂)₈Me (2-p) |
| | | | | | | | —Me (5-p) |
| No. 25 | NH | C | CH | CH | C | — | —(CH₂)₆Me (2-p) |
| | | | | | | | —(CH₂)₆Me (5-p) |

*Note: p means the position

The results of the ¹H NMR spectroscopic analysis of the foregoing compounds will be listed in the following Table 2.

TABLE 2

| Comp. No. | ¹H NMR {400 MHz, CDCl₃/TMS, • (ppm)} |
|---|---|
| No. 1 | 1.29-1.37 (6H, m), 1.52-1.57 (2H, m), 1.74-1.81 (2H, m), |
| | 3.63 (2H, t, J = 6.6 Hz), 3.92 (2H, t, J = 7.1 Hz), 6.90 |
| | (1H, s), 7.04 (1H, s), 7.46 (1H, s) |
| No. 2 | 1.97-2.04 (2H, m), 3.63 (2H, t, J = 5.8 Hz), 4.13 (2H, t, |
| | J = 6.8 Hz), 6.93 (1H, s), 7.04 (1H, s), 7.46 (1H, s) |
| No. 3 | 1.35-1.42 (2H, m), 1.56-1.63 (2H, m), 1.78-1.86 (2H, m), |
| | 3.64 (2H, t, J = 6.6 Hz), 3.95 (2H, t, J = 7.1 Hz), 6.91 |
| | (1H, s), 7.04 (1H, s), 7.46 (1H, s) |
| No. 4 | 1.28-1.44 (4H, m), 1.52-1.59 (2H, m), 1.75-1.82 (2H, m), |
| | 3.63 (2H, t, J = 6.4 Hz), 3.93 (2H, t, J = 7.1 Hz), 6.90 |
| | (1H, s), 7.04 (1H, s), 7.45 (1H, s) |
| No. 5 | 1.25-1.36 (8H, m), 1.52-1.59 (2H, m), 1.74-1.81 (2H, m), |
| | 3.63 (2H, t, J = 6.6 Hz), 3.92 (2H, t, J = 7.1 Hz), 6.90 |
| | (1H, s), 7.05 (1H, s), 7.46 (1H, s) |
| No. 6 | 1.27-1.33 (12H, m), 1.52-1.59 (2H, m), 1.74-1.79 (2H, m), |
| | 3.62 (2H, t, J = 6.8 Hz), 3.92 (2H, t, J = 7.1 Hz), 6.90 |
| | (1H, s), 7.03 (1H, s), 7.45 (1H, s) |
| No. 7 | 1.33-1.41 (2H, m), 1.56-1.63 (2H, m), 1.77-1.84 (2H, m), |
| | 3.32 (3H, s), 3.35 (2H, t, J = 6.4 Hz), 3.94 (2H, t, |
| | J = 7.1 Hz), 6.91 (1H, s), 7.06 (1H, s), 7.47 (1H, s) |
| No. 8 | 1.27-1.42 (4H, m), 1.52-1.59 (2H, m), 1.75-1.82 (2H, m), |
| | 3.32 (3H, s), 3.35 (2H, t, J = 6.3 Hz), 3.93 (2H, t, |
| | J = 7.1 Hz), 6.90 (1H, s), 7.06 (1H, s), 7.47 (1H, s) |
| No. 9 | 1.25-1.36 (6H, m), 1.52-1.58 (2H, m), 1.74-1.81 (2H, m), |
| | 3.33 (3H, s), 3.35 (2H, t, J = 6.6 Hz), 3.92 (2H, t, |
| | J = 7.1 Hz), 6.90 (1H, s), 7.06 (1H, s), 7.47 (1H, s) |
| No. 10 | 1.31 (8H, br), 1.52-1.57 (2H, m), 1.73-1.81 (2H, m), |
| | 3.33 (3H, s), 3.36 (2H, t, J = 6.6 Hz), 3.92 (2H, t, |
| | J = 7.1 Hz), 6.90 (1H, s), 7.06 (1H, s), 7.47 (1H, s) |
| No. 11 | 1.26-1.36 (6H, m), 1.52-1.58 (2H, m), 1.73-1.80 (2H, m), |
| | 3.63 (2H, t, J = 6.6 Hz), 3.86 (2H, t, J = 7.1 Hz), 6.13 |
| | (2H, dd, J = 2.0 Hz, J = 2.0 Hz), 6.64 (2H, dd, J = 2.0 Hz, |
| | J = 2.0 Hz) |
| No. 12 | 1.30-1.39 (6H, m), 1.54-1.59 (2H, m), 1.89-1.96 (2H, m), |
| | 3.61-3.64 (2H, m), 4.39 (2H, t, J = 7.1 Hz), 7.54 (1H, s), |
| | 7.70 (1H, s) |
| No. 13 | 1.28-1.31 (12H, m), 1.53-1.59 (2H, m), 1.89-1.93 (2H, m), |
| | 3.62-3.66 (2H, m), 4.39 (2H, t, J = 7.1 Hz), 7.53 (1H, s), |
| | 7.71 (1H, s) |
| No. 14 | 1.28-1.38 (6H, m), 1.52-1.59 (2H, m), 1.86-1.93 (2H, m), |
| | 3.62-3.64 (2H, m), 4.17 (2H, t, J = 7.1 Hz), 7.94 (1H, s), |
| | 8.05 (1H, s) |
| No. 15 | 1.22-1.38 (12H, m), 1.52-1.59 (2H, m), 1.85-1.92 (2H, m), |
| | 3.61-2-3.66 (2H, m), 4.16 (2H, t, J = 7.1 Hz), 7.94 (1H, s), |
| | 8.04 (1H, s) |
| No. 16 | 1.28-1.37 (6H, m), 1.51-1.56 (2H, m), 1.83-1.90 (2H, m), |
| | 3.63 (2H, t, J = 6.6 Hz), 4.12 (2H, t, J = 7.3 Hz), 6.23 |
| | (1H, dd, J = 2.0 Hz, J = 1.2 Hz), 7.37 (1H, d, J = 2.0 Hz), |
| | 7.50 (1H, d, J = 1.2 Hz) |
| No. 17 | 1.49-1.91 (10H, m), 2.07-2.14 (2H, m), 4.04-4.16 (1H, m), |
| | 6.94 (1H, s), 7.03 (1H, s), 7.52 (1H, s) |
| No. 18 | 1.32-1.37 (6H, m), 1.52-1.57 (2H, m), 1.68-1.76 (2H, m), |
| | 2.37 (3H, s), 3.63 (2H, t, J = 6.6 Hz), 3.81 (2H, t, |
| | J = 7.1 Hz), 6.80 (1H, d, J = 1.2 Hz), 6.89 (1H, d, |
| | J = 1.2 Hz) |
| No. 19 | 0.88 (3H, t, J = 6.6 Hz), 1.26-1.35 (22H, m), 1.53-1.59 |
| | (2H, m), 1.69-1.79 (4H, m), 2.62 (2H, t, J = 8.1 Hz), 3.64 |
| | (2H, t, J = 6.3 Hz), 3.81 (2H, t, J = 7.3 Hz), 6.79 (1H, d, |
| | J = 1.2 Hz), 6.93 (1H, d, J = 1.2 Hz) |
| No. 20 | 0.99 (3H, t, J = 7.3 Hz), 1.38 (3H, t, J = 7.3 Hz), |
| | 1.77-1.87 (2H, m), 2.79 (2H, t, J = 7.6 Hz), 4.39 (2H, q, |
| | J = 7.3 Hz), 8.15 (1H, s) |
| No. 21 | 0.88 (3H, t, J = 6.8 Hz), 1.22-1.40 (15H, m), |
| | 1.74-1.81 (2H, m), 2.81 (2H, t, J = 7.6 Hz), 4.39 (2H, t, |
| | J = 7.1 Hz), 8.13 (1H, s) |
| No. 22 | 0.88 (3H, t, J = 6.8 Hz), 1.26-1.32 (12H, m), |
| | 1.75-1.82 (2H, m), 2.84 (2H, t, J = 7.6 Hz), 8.22 (1H, s) |
| No. 23 | 0.88 (3H, t, J = 6.6 Hz), 1.26-1.33 (12H, m), |
| | 1.72-1.78 (2H, m), 2.76 (2H, t, J = 7.6 Hz), 7.01 (1H, s), |
| | 7.55 (1H, s) |
| No. 24 | 0.88 (3H, t, J = 6.6 Hz), 1.26-1.32 (12H, m), |
| | 1.69-1.77 (2H, m), 2.27 (3H, s), 2.69 (2H, t, J = 7.6 Hz), |
| | 6.58 (1H, s) |
| No. 25 | 0.88 (6H, t, J = 6.8 Hz), 1.27-1.38 (16H, m), |
| | 1.55-1.63 (4H, m), 2.54 (4H, t, J = 7.8 Hz), 5.77 (2H, s), |
| | 7.56 (1H, br) |

(In the results of the ¹H NMR spectroscopic analysis, the signal states are expressed as follows: s (singlet); d (doublet); t (triplet); q (quadruplet); quint (quintet); m (multiplet); br (broad) and so forth.

### Example 1

### Preparation of 7-imidazol-1-yl-heptan-1-ol represented by the following formula (Compound No. 1)

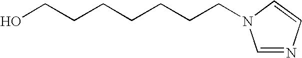

To 7-bromo-1-heptanol (2.0 g, 10.3 mM), there was added 30 mL of dehydrated dimethylformamide as a reaction solvent and then imidazole (837 mg, 12.3 mM) was further added to the resulting mixture. To the resulting mixture, there was added sodium hydride (50 to 72%, 451 mg), followed by the stirring thereof at room temperature for 30 minutes, and the subsequent heating, with stirring, at 125° C. for 7 hours. After the completion of the heating with stirring, 100 mL of purified water was added to the reaction system and then the system was extracted twice with 100 mL of chloroform. The extracts thus recovered were combined together, followed by the addition of sodium sulfate to the combined extract, the drying and the filtration of the same, and the removal of the solvent through distillation. After the removal of the solvent through distillation, the resulting residue was purified by the silica gel column chromatography (elution was carried out using ethyl acetate and 5:1 mixed solvent of ethyl acetate and methanol) to thus give the title compound (1.76 g, 94.2%) as a pale yellow and transparent liquid.

¹H-NMR (400 MHz:CDCl₃) δ:1.29-1.37(6H, m), 1.52-1.57(2H, m), 1.74-1.81(2H, m), 3.63(2H, t, J=6.6 Hz), 3.92(2H, t, J=7.1 Hz), 6.90(1H,s), 7.04(1H,s), 7.46(1H,s).

The compounds (II) represented by Formula (I) specifically synthesized herein are those having each corresponding structure summarized in the following Table 3. In this respect, however, the number used for specifying the position of each specific atom constituting the 6-membered ring (1-position to 6-position) shown in Table 1 is herein simply given for convenience and it is not controlled by the IUPAC nomenclature at all.

TABLE 3

(II)

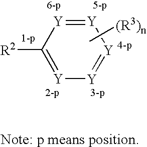

| Comp. | Y | Y | Y | Y | Y | R² | R³ |
|---|---|---|---|---|---|---|---|
| No. | (2-p) | (3-p) | (4-p) | (5-p) | (6-p) | (1-p) | (2-p to 6-p) |
| No.26 | CH | CH | N | CH | CH | —(CH₂)₆Me | — |
| No.27 | CH | N | CH | CH | CH | —(CH₂)₆Me | — |
| No.28 | N | CH | CH | CH | CH | —(CH₂)₆Me | — |
| No.29 | CH | CH | N | CH | CH | —O(CH₂)₆Me | — |
| No.30 | CH | N | CH | CH | CH | —O(CH₂)₆Me | — |
| No.31 | N | CH | CH | CH | CH | —O(CH₂)₆Me | — |
| No.32 | CH | CH | N | CH | CH | —O(CH₂)₇OH | — |
| No.33 | CH | CH | N | CH | CH | —O(CH₂)₅OH | — |
| No.34 | CH | CH | N | CH | CH | —O(CH₂)₃OH | — |
| No.35 | CH | CH | N | CH | CH | —O(CH₂)₂OH | — |
| No.36 | CH | CH | N | CH | CH | —CH₂(OCH₂CH₂)₂OH | — |
| No.37 | CH | CH | N | CH | CH | —CH₂OCH₂CH₂OH | — |

*Note: p means the position. This is also true for the following description.

The results of the ¹H NMR spectroscopic analysis of the foregoing compounds will be listed in the following Table 4.

TABLE 4

| Comp. No. | ¹H NMR {400 MHz, CDCl₃/TMS, • (ppm)} |
|---|---|
| No. 26 | 0.88 (3H, t, J = 6.8 Hz), 1.28-1.32 (8H, m), 1.60-1.64 |
| | (2H, m), 2.60 (2H, t, J = 7.6 Hz), 7.10 (2H, d, J = 5.6 Hz), |
| | 8.48 (2H, d, J = 5.6 Hz) |
| No. 27 | 0.88 (3H, t, J = 6.8 Hz), 1.27-1.34 (8H, m), 1.58-1.65 |
| | (2H, m), 2.60 (2H, t, J = 7.6 Hz), 7.18-7.21 (1H, m), |
| | 7.47-7.50 (1H, m), 8.42-8.44 (2H, m) |
| No. 28 | 0.87 (3H, t, J = 6.8 Hz), 1.26-1.42 (8H, m), 1.70-1.79 |
| | (2H, m), 2.78 (2H, t, J = 7.8 Hz), 7.07-7.11 (1H, m), |
| | 7.14 (1H, d, J = 6.8 Hz), 7.56-7.60 (1H, m), |
| | 8.51-8.53 (1H, m) |
| No. 29 | 0.90 (3H, t, J = 6.8 Hz), 1.28-1.47 (8H, m), 1.76-1.82 |
| | (2H, m), 4.00 (2H, t, J = 6.6 Hz), 6.79 (2H, dd, J = 4.8 Hz, |
| | J = 1.6 Hz), 8.41 (2H, dd, J = 4.8 Hz, J = 1.6 Hz) |
| No. 30 | 0.90 (3H, t, J = 6.8 Hz), 1.29-1.48 (8H, m), 1.78-1.82 |
| | (2H, m), 3.99 (2H, t, J = 6.4 Hz), 7.17-7.20 (2H, m), |
| | 8.19-8.21 (1H, m), 8.30-8.31 (1H, m) |
| No. 31 | 0.87 (3H, t, J = 7.1 Hz), 1.26-1.34 (8H, m), 1.72-1.76 |
| | (2H, m), 3.91 (2H, t, J = 7.3 Hz), 6.13-6.16 (1H, m), |
| | 6.54-6.57 (1H, m), 7.24-7.32 (2H, m) |
| No. 32 | 1.39-1.69 (8H, m), 1.77-1.84 (2H, m), 3.65 (2H, t, |
| | J = 6.4 Hz), 4.00 (2H, t, 6.6 Hz), 6.78-6.80 (2H, m), |
| | 8.40-8.41 (2H, m) |
| No. 33 | 1.53-1.68 (4H, m), 1.82-1.87 (2H, m), 3.69 (2H, t, |
| | J = 6.1 Hz), 4.02 (2H, t, 6.4 Hz), 6.79 (2H, dd, J = 4.8 Hz, |
| | J = 1.5 Hz), 8.41 (2H, dd, J = 4.8 Hz, J = 1.5 Hz) |
| No. 34 | 2.00-2.07 (2H, m), 3.84 (2H, t, J = 5.9 Hz), 4.13 |
| | (2H, t, 6.1 Hz), 6.75 (2H, dd, J = 4.8 Hz, J = 1.5 Hz), |
| | 8.33 (2H, dd, J = 4.8 Hz, J = 1.5 Hz) |
| No. 35 | 4.00 (2H, t, J = 4.4 Hz), 4.14 (2H, t, 4.4 Hz), 6.83 |
| | (2H, dd, J = 4.7 Hz, J = 1.5 Hz), 8.45 (2H, dd, J = 4.7 Hz, |
| | J = 1.5 Hz) |
| No. 36 | 3.61-3.78 (8H, m), 4.60 (2H, s), 7.26-7.28 (2H, m), 8.58 |
| | (2H, dd, J = 4.4 Hz, J = 1.5 Hz) |
| No. 37 | 3.65 (2H, t, J = 4.6 Hz), 3.82 (2H, t, 4.6 Hz), 4.59 (2H, s), |
| | 7.25-7.28 (2H, m), 8.56 (2H, dd, J = 4.5 Hz, J = 1.5 Hz) |

(In the results of the ¹H NMR spectroscopic analysis, the signal states or shapes are expressed as follows: s (singlet); d (doublet); t (triplet); q (quadruplet); quint (quintet); m (multiplet); br (broad) and so forth.

### Preparation of Pharmaceutical Preparations Using Drug-Permeation Promoter and Evaluation of Permeation-Improving Effect of the Same

Now, examples of pharmaceutical preparations and the Drug-Permeation Improving effect thereof will be detailed below, but the scope of the present invention is not restricted to these specific examples of pharmaceutical preparations, at all.

### Example 2

### Pharmaceutical Preparation A as a Trial-Produced One

A nail lacquer type treating agent for external use, which is used in the treatment of ringworm of the nail, while making use of 7-imidazol-1-yl-heptan-1-ol (Compound No. 1) as a drug-permeation promoter. In this respect, butenafine hydrochloride was used as a drug component.

The pharmaceutical preparation A had a composition specified below. The pharmaceutical preparation comprised butenafine in the form of butenafine hydrochloride in an amount of 5% (w/v).

| | | |
|---|---|---|
| Compound No. 1 | 0.500 | g |
| Butenafine hydrochloride | 0.279 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 3

### Pharmaceutical Preparation B as a Trial-Produced One

A nail lacquer type treating agent for external use, which is used in the treatment of ringworm of the nail, while making use of 5-(pyridin-4-yloxy)-pentan-1-ol (Compound No. 33) as a drug-permeation promoter. In this respect, butenafine hydrochloride was used as a basic effective component.

The pharmaceutical preparation B had a composition specified below. The pharmaceutical preparation comprised butenafine in the form of butenafine hydrochloride in an amount of 5% (w/v).

| | | |
|---|---|---|
| Compound No. 33 | 0.500 | g |
| Butenafine hydrochloride | 0.279 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 4

### Pharmaceutical Preparation C as a Trial-Produced One

A nail lacquer type treating agent for external use, which is used in the treatment of ringworm of the nail, while making use of 4-heptyloxy-pyridine (Compound No. 29) as a drug-permeation promoter. In this respect, butenafine hydrochloride was used as a basic effective component.

The pharmaceutical preparation C had a composition specified below. The pharmaceutical preparation comprised butenafine in the form of butenafine hydrochloride in an amount of 5% (w/v).

| | | |
|---|---|---|
| Compound No. 29 | 0.500 | g |
| Butenafine hydrochloride | 0.279 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 5

### Pharmaceutical Preparation D as a Trial-Produced One

A nail lacquer type treating agent for external use, which is used in the treatment of ringworm of the nail, while making use of 3-heptyloxy-pyridine (Compound No. 30) as a drug-permeation promoter. In this respect, butenafine hydrochloride was used as a basic effective component. The pharmaceutical preparation D had a composition specified below. The pharmaceutical preparation comprised butenafine in the form of butenafine hydrochloride in an amount of 5% (w/v).

| | | |
|---|---|---|
| Compound No. 30 | 0.500 | g |
| Butenafine hydrochloride | 0.279 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 6

### Pharmaceutical Preparation E as a Trial-Produced One

A nail lacquer type treating agent for external use, which is used in the treatment of ringworm of the nail, while making use of 2-heptyloxy-pyridine (Compound No. 31) as a drug-permeation promoter. In this respect, butenafine hydrochloride was used as a basic effective component. The pharmaceutical preparation E had a composition specified below. The pharmaceutical preparation comprised butenafine in the form of butenafine hydrochloride in an amount of 5% (w/v).

| | | |
|---|---|---|
| Compound No. 31 | 0.500 | g |
| Butenafine hydrochloride | 0.279 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 7

### Pharmaceutical Preparation F as a Trial-Produced One

A nail lacquer type treating agent for external use, which is used in the treatment of ringworm of the nail, while making use of 7-pyrrol-1-yl-hepten-1-ol (Compound No. 11) as a drug-permeation promoter. In this respect, butenafine hydrochloride was used as a basic effective component.

The pharmaceutical preparation F had a composition specified below. The pharmaceutical preparation comprised butenafine in the form of butenafine hydrochloride in an amount of 5% (w/v).

| | | |
|---|---|---|
| Compound No. 11 | 0.500 | g |
| Butenafine hydrochloride | 0.279 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 8

### Pharmaceutical Preparation G as a Trial-Produced One

A nail lacquer type treating agent for external use, which is used in the treatment of ringworm of the nail, while making use of 7-imidazol-1-yl-heptan-1-ol (Compound No. 1) as a drug-permeation promoter. In this respect, butenafine hydrochloride was used as a basic effective component. The pharmaceutical preparation G had a composition specified below. The pharmaceutical preparation comprised butenafine in the form of butenafine hydrochloride in an amount of 15% (w/v).

| | | |
|---|---|---|
| Compound No. 1 | 0.500 | g |
| Butenafine hydrochloride | 0.836 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 9

### Pharmaceutical Preparation H as a Trial-Produced One

A nail lacquer type treating agent for external use, which is used in the treatment of ringworm of the nail, while making use of 7-imidazol-1-yl-heptan-1-ol (Compound No. 1) as a drug-permeation promoter. In this respect, butenafine hydrochloride was used as an effective component. The pharmaceutical preparation H had a composition specified below. The pharmaceutical preparation comprised terbinafine in the form of terbinafine hydrochloride in an amount of 15% (w/v).

| | | |
|---|---|---|
| Compound No. 1 | 0.500 | g |
| Terbinafine hydrochloride | 0.844 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 10

### Pharmaceutical Preparation I as a Trial-Produced One

A nail lacquer type treating agent for external use, which is used in the treatment of ringworm of the nail, while making use of 7-imidazol-1-yl-heptan-1-ol (Compound No. 1) as a drug-permeation promoter. In this respect, amorolfine hydrochloride was used as an effective component.

The pharmaceutical preparation I had a composition specified below. The pharmaceutical preparation comprised amorolfine in the form of amorolfine hydrochloride.

| | | |
|---|---|---|
| Compound No. 1 | 0.500 | g |
| Amorolfine hydrochloride | 0.836 | g |
| EUDRAGIT L100 | 0.625 | g |
| Butyl acetate | 0.250 | g |
| Ethyl acetate | 0.750 | g |
| Anhydrous ethanol | ad. 5 | mL |

### Example 11

### Evaluation of Permeation-Improving Effect of Pharmaceutical Preparations Prepared Using Drug-Permeation Promoter

Now, the drug-permeation improving effect of pharmaceutical preparations will be detailed below, while taking specific examples, but the scope of the present invention is not restricted to these specific examples, at all.

### Test Methods for Evaluating the Effect

### Method for Evaluating the Amount of Drug Permeated Through the Nail

First of all, to 70% propylene glycol in purified water, there was added 1% Bacto Agar (available from Becton•Dickinson Company) to thus prepare agarose-agar, then 1 mL each of the resulting agarose-agar was dispensed into vials (inner diameter: 24 mm; height: 55 mm) each provided with a screw cap and then the agarose-agar was solidified in such a manner that the surface of the agarose-agar in each vial was even or flat. Then, the human nail was precisely cut to give a piece of 4×4 mm, an O-ring of silicone having an outer diameter of 2.5 mm was adhered to the center on the upper surface of the nail specimen with a silicone type adhesive and 5 μL of the pharmaceutical preparation prepared above was applied onto the nail surface encircled with the O-ring. The nail specimen was gently placed on the agarose-agar such that the O-ring turned upward and that the upper portion of the nail and the O-ring never came into close contact with the agarose-agar. After placing the nail specimen on the agarose-agar, it was allowed to stand over 5 days at a temperature of 37° C., followed by the removal of the nail, the addition of 1N sodium hydroxide solution to the agarose-agar to thus make the agar basic, the extraction of the antibiotic agent permeated into the agarose-agar through the nail agar twice with ethyl acetate and the determination of the amount of the antibiotic agent as the effective component of the preparation by the HPLC technique.

The following Tables 5, 6, 7 and 8 shows the amounts of butenafine hydrochloride, terbinafine hydrochloride and amorolfine hydrochloride which permeated through the nail and determined according to the aforementioned test method.

The amount of butenafine hydrochloride incorporated into the pharmaceutical preparation containing butenafine in the form of butenafine hydrochloride (5% (w/v)), which could permeate into the agarose-agar through the nail (the concentration of the compound (permeation-improving agent) was set at a level of 10% (w/v))

TABLE 5

| Pharmaceutical | The No. of Comp. | Amt. of Drug |
|---|---|---|
| Preparation | Added to the | Penetrable through the |
| (trial-prepared) | Preparation | nail*² |
| Trial Preparation A | No. 1 | 14.4 μg |
| Trial Preparation B | No. 33 | 12.1 μg |
| Trial Preparation C | No. 29 | 6.44 μg |
| Trial Preparation D | No. 30 | 2.28 μg |
| Trial Preparation E | No. 31 | 2.35 μg |
| Trial Preparation F | No. 11 | 3.29 μg |
| Placebo a*¹ | Free of any Promoter | 1.83 μg |

*¹A pharmaceutical preparation obtained by the removal of only the permeation-improving agent from the composition of the trial pharmaceutical preparation A;
*²The amount of butenafine quantitatively determined as expressed in terms of the amount of the butenafine hydrochloride.

The amount of butenafine hydrochloride incorporated into the pharmaceutical preparation containing butenafine in the form of butenafine hydrochloride (15% (w/v)), which could permeate into the agarose-agar through the nail (the concentration of the compound (permeation-improving agent) was set at a level of 10% (w/v))

TABLE 6

| Pharmaceutical | The No. of Comp. | Amt. of Drug |
|---|---|---|
| Preparation | Added to the | Penetrable through the |
| (trial-prepared) | Preparation | nail*⁴ |
| Trial Preparation G | No. 1 | 29.5 μg |
| Placebo g*³ | Free of any Promoter | 0.89 μg |

*³A pharmaceutical preparation obtained by the removal of only the permeation-improving agent from the composition of the trial pharmaceutical preparation G;
*⁴The amount of butenafine quantitatively determined as expressed in terms of the amount of the butenafine hydrochloride.

The amount of terbinafine hydrochloride incorporated into the pharmaceutical preparation containing terbinafine in the form of terbinafine hydrochloride (15% (w/v)), which could permeate into the agarose-agar through the nail (the concentration of the compound (permeation-improving agent) was set at a level of 10% (w/v))

TABLE 7

| Pharmaceutical | The No. of Comp. | Amt. of Drug |
|---|---|---|
| Preparation | Added to the | Penetrable through the |
| (trial-prepared) | Preparation | nail*⁶ |
| Trial Preparation H | No. 1 | 35.4 μg |
| Placebo h*⁵ | Free of any Promoter | 2.31 μg |

*⁵A pharmaceutical preparation obtained by the removal of only the permeation-improving agent from the composition of the trial pharmaceutical preparation H;
*⁶The amount of terbinafine quantitatively determined as expressed in terms of the amount of the terbinafine hydrochloride.

The amount of amorolfine hydrochloride incorporated into the pharmaceutical preparation containing amorolfine in the form of amorolfine hydrochloride (15% (w/v)), which could permeate into the agarose-agar through the nail (the concentration of the compound (permeation-improving agent) was set at a level of 10% (w/v))

TABLE 8

| Pharmaceutical | The No. of Comp. | Amt. of Drug |
|---|---|---|
| Preparation | Added to the | Penetrable through the |
| (trial-prepared) | Preparation | nail*⁸ |
| Trial Preparation I | No. 1 | 61.5 μg |
| Placebo i*⁷ | Free of any Promoter | 2.42 μg |

*⁷A pharmaceutical preparation obtained by the removal of only the permeation-improving agent from the composition of the trial pharmaceutical preparation I;
*⁸The amount of amorolfine quantitatively determined as expressed in terms of the amount of the amorolfine hydrochloride.

The foregoing test results clearly indicate that the various kinds of antibiotic-containing pharmaceutical preparations which comprise the permeation-improving agent according to the present invention show distinct drug-permeation improving effect in the foregoing test for determining the amount of the antibiotic agent permeated through the human nail specimen. For instance, in the permeated amount-determining test (Table 5) using a pharmaceutical preparation which contains 5% (w/v) butenafine hydrochloride as a butenafine effective component and Compound No. 1 as the permeation-improving agent, the butenafine hydrochloride-permeation improving effect observed for the trial pharmaceutical preparation A comprising 10% Compound No. 1 as a permeation-improving agent was found to be about 7.9 times higher than that observed for the placebo a free of Compound No. 1. Similarly, the butenafine hydrochloride-permeation improving effect observed for the trial pharmaceutical preparation B comprising 10% of Compound No. 33 as a permeation-improving agent was found to be about 6.6 times higher than that observed for the placebo a.

The drug-permeation improving effect of Compound No. 1 is conspicuous, in particular, when the concentration of the antibiotic agent is high. More specifically, in the permeated amount-determining test (Table 6) using a pharmaceutical preparation which contains 15% (w/v) butenafine hydrochloride as a butenafine effective component and Compound No. 1 as the permeation-improving agent, the butenafine hydrochloride-permeation improving effect observed for the trial pharmaceutical preparation G comprising 10% Compound No. 1 as a permeation-improving agent was found to be about 33 times higher than that observed for the placebo g free of Compound No. 1.

Similarly, in the permeated amount-determining test (Table 7) using a trial pharmaceutical preparation H which contains 15% (w/v) terbinafine hydrochloride as a terbinafine effective component and Compound No. 1 as the permeation-improving agent, the terbinafine hydrochloride-permeation improving effect observed for the trial pharmaceutical preparation H was found to be about 15 times higher than that observed for the placebo h free of Compound No. 1, while in the permeated amount-determining test (Table 8) using a trial pharmaceutical preparation I which contains 15% (w/v) terbinafine hydrochloride as a terbinafine effective component and Compound No. 1 as the permeation-improving agent, the terbinafine hydrochloride-permeation improving effect observed for the trial pharmaceutical preparation I was found to be about 25 times higher than that observed for the placebo i free of Compound No. 1. As has been discussed above, the compound of the present invention shows an excellent drug-permeation promoter effect and accordingly, it can be proved that the compound is highly useful in the prevention and treatment of diseases relating to the nail.

### INDUSTRIAL APPLICABILITY

As has been discussed above in detail, the present invention herein provides novel compounds each having a strong effect of improving the permeation of any therapeutic agent into the nail and therefore, the compound is quite useful in the prevention and treatment of such ringworm as infectious diseases, inflammation and allergy including ringworm. Up to now, it has been quite difficult to treat the diseases of the nail with external preparations, but it would be expected that the treatment thereof with external preparations if using the compound of the present invention. Accordingly, the compound of the present invention would be highly valuable in its applications.

What is claimed is:

1. A drug-permeation promoter consisting essentially of a compound represented by the following Formula (I) or a pharmaceutically acceptable salt thereof: 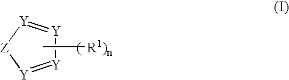
wherein in Formula (I),
Z represents a secondary nitrogen atom (—NH—), an oxygen atom (—O—) or a sulfur atom (—S—);
Y represents a tertiary nitrogen atom (—N═) or a tertiary carbon atom (—═);
R¹ represents a C1 to C15 saturated or unsaturated hydrocarbon group [except for the compounds of Formula (I) in which R¹ is a saturated hydrocarbon group represented by CH₃CₘH₂ₘ wherein m is a number ranging from 5 to 14 and it is directly bonded to the nitrogen atom on the 5-membered ring in Formula (I)]; and
n is a number ranging from 1 to 5.

2. A drug-permeation promoter comprising a compound represented by the following general formula (II) or a pharmaceutically acceptable salt thereof: 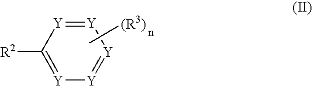
wherein in Formula (II),
R² and R³ independently represent a C1 to C15 saturated or unsaturated hydrocarbon group or a C1 to C15 saturated or unsaturated hydrocarbon group connected to the 6-membered heterocyclic ring through an oxygen atom;
Y represents a tertiary nitrogen atom (—N═) or a tertiary carbon atom (—C═); and
n is a number ranging from 0 to 5.

3. A nail-treating agent for external use comprising the drug-permeation promoter as set forth in claim 1 and a drug component.

4. A nail-treating agent for external use comprising the drug-permeation promoter as set forth in claim 2 and a drug component.

5. The nail-treating agent for external use as set forth in claim 3, wherein the drug component is a member selected from the group consisting of butenafine, terbinafine and amorolfine.

6. The nail-treating agent for external use as set forth in claim 4, wherein the effective component is a member selected from the group consisting of butenafine, terbinafine and amorolfine.
